(19) United States
(12) Patent Application Publication
(10) Pub. No.: US 2015/0231614 A1
(43) Pub. Date: Aug. 20, 2015

(54) METHOD FOR PRE-TREATING A CATALYST COMPOSITION

(71) Applicant: SAUDI BASIC INDUSTRIES CORPORATION, RIYADH (SA)

(72) Inventor: Suman Kumar Jana, Bangalore (IN)

(21) Appl. No.: 14/421,363

(22) PCT Filed: Aug. 13, 2013
(86) PCT No.: PCT/IB2013/056622

§ 371 Date: Feb. 12, 2015

(30) Foreign Application Priority Data
Aug. 14, 2012 (EP) 12005876.3

(51) Int. Cl. B01J 29/70; B01J 29/40; C07C 2/12; B01J 29/08; B01J 29/85; B01J 37/10; B01J 29/18

(52) U.S. Cl. CPC B01J 29/7007; B01J 37/10; B01J 29/40; B01J 29/18; B01J 29/084; B01J 29/85; C07C 2/12; C07C 2529/85; C07C 2529/18; C07C 2529/08; C07C 2529/40; C07C 2529/70

(57) ABSTRACT

The present invention relates to a method for pre-treating a catalyst composition comprising contacting a medium pore aluminosilicate zeolite with an inert gas comprising water vapour or alcohol vapour at a temperature between 30° C. and the boiling temperature of water or the alcohol.

### CROSS-REFERENCE TO RELATED APPLICATIONS

This application is a National Stage Filing of International Application No. PCT/IB2013/056622; filed on Aug. 13, 2013, which claims priority to EP Patent Application No. 12005876.3; filed Aug. 14, 2012, both of which are incorporated herein by reference in their entirety.

### BACKGROUND

The present invention relates to a method for pre-treating a catalyst composition. The invention further relates to a process for the dimerization of olefins or a mixture of olefins and paraffins using the pre-treated catalyst composition.

A variety of octane boosters is on the market. Currently, the most popular octane booster is methyl tertiary butyl ether (MTBE). However, because of its hazardous nature, it is predicted that the worldwide consumption of MTBE will gradually decrease. A promising candidate for replacing MTBE is isooctane. Isooctane has similar properties to MTBE, exhibits high octane number and also has very good environmental properties.

Isooctane is typically produced by dimerization of isobutene followed by hydrogenation. A few technologies are available for commercialization for the selective production of isooctenes from isobutene based on solid macro-porous acidic resin or phosphoric acid catalyst. These technologies are based on solid macro-porous acidic resin or phosphoric acid catalyst and require additive(s) and/or hydrating agent(s) together with the catalyst to obtain isooctenes selectively. However, the presence of additive(s)/hydrating agent(s) in the feed stream contributes to the formation of some amount of unwanted product in the product stream.

Catalysis Today, 100 (2005) 463 discloses catalyst H-ZSM-5 (having Si/Al ratios of 15 and 25) for isobutene dimerization under liquid-phase conditions (40° C., 10 bar pressure and n-butane as diluent). Although the catalyst shows high initial conversion, it deactivates very rapidly. The initial conversion of ˜65% drops down to <2% within 1 h of reaction, hence it is not suitable for industrial scale dimerization of isobutene.

EP 1167326 A1 discloses the dimerization of isobutene using a catalyst system comprising a zeolite with TON-type structure. The catalyst was unable to show the desired product selectivity for a long time. The dimer selectivity decreased from 72% to 54% within 4 h of reaction. The catalyst also got deactivated fast. Isobutene conversion decreased from 98% to 84% within 4 h of reaction.

WO 2004/080935 A1 discloses medium pore zeolites. H-ZSM-5, H-ZSM-22, H-ZSM-23, Ferrierite having Al content ˜0.1 to 5 wt % are reported for isobutene dimerization under high pressure reaction conditions. However, these catalysts deactivate fast and hence require frequent regeneration for long reaction process.

WO09027582 discloses a process for oligomerizing olefinic, lower hydrocarbons using an acidic catalyst selected from the group of natural and synthetic zeolites or from the mesoporous aluminosilicates. Zeolite is selected from the group consisting of ZSM-5, ZSM-22, ZSM-23, ferrierite and ion-exchanged zeolites prepared therefrom.

WO200480935 discloses a process for dimerizing lower, olefinic hydrocarbons with a medium pore zeolite under process conditions allowing selective dimerization. The olefinic hydrocarbon feedstock is contacted with an acid catalyst at conditions in which at least a part of the olefins dimerizes. The effluent from the reaction zone is conducted to the separation zone where dimerized reaction product is separated from said effluent.

U.S. Pat. No. 3,325,465 discloses a process for dimerizing isobutene using 13× molecular sieve wherein the 95.9% of sodium ion is counter ion exchanged with cobalt ion.

The major disadvantages of the known prior art are low isooctene selectivity and low catalyst stability. Also the use of ion-exchange resin catalysts requires cumbersome catalyst regeneration, which leads to the generation of more effluents.

### SUMMARY

It is an object of the present invention to provide an improved catalyst. It is a further object of the present invention to provide an improved process for the dimerization of olefins or a mixture of olefins and paraffins.

Accordingly, the present invention provides a method for pre-treating a catalyst composition comprising contacting a medium pore aluminosilicate zeolite with an inert gas comprising water vapour or alcohol vapour at a temperature between 30° C. and the boiling temperature of water or the alcohol.

### DETAILED DESCRIPTION

It will be appreciated that the feature “contacting a medium pore aluminosilicate zeolite with an inert gas comprising water vapour or alcohol vapour at a temperature . . . ” is understood to mean that the medium pore aluminosilicate zeolite having the specified temperature is contacted with the inert gas comprising water vapour or alcohol vapour. Accordingly, in the method of the present invention, the temperature of the medium pore aluminosilicate zeolite is controlled to a temperature between 30° C. and the boiling temperature of water or the alcohol and is contacted with the inert gas comprising water vapour or alcohol vapour. The method of the present invention may also be described as a method for pre-treating a catalyst composition comprising contacting a medium pore aluminosilicate zeolite with an inert gas comprising water vapour or alcohol vapour, wherein the zeolite has a temperature between 30° C. and the boiling temperature of water or the alcohol.

The temperature of the zeolite which comes into contact with the inert gas is herein sometimes referred as the contact temperature.

It is noted that U.S. Pat. No. 4,044,065 discloses a method for preparing a phosphorus-containing zeolite in which the phosphorus-containing zeolite is contacted with water vapour prior to its use as a catalyst. In example 2 of U.S. Pat. No. 4,044,065, the phosphorus-containing zeolite is placed in a quartz tube with a thermowell in the center and heated to 130-140° C. and nitrogen saturated with water at 30-50° C. is passed through the tube for 20 hours. U.S. Pat. No. 4,044,065 does not disclose contacting a zeolite having a temperature between 30° C. and the boiling temperature of water or the alcohol with an inert gas comprising water vapour or the alcohol vapour.

According to the pre-treatment method of the present invention, the relatively strong acid sites of zeolite catalyst is reduced through control adsorption of water or alcohol molecule on the acidic sites. It was found that the hydrophilic nature of the alcohol molecule and water molecule results in the adsorption on the acidic sites. This results in that the severity of the dimerization reaction is minimized. The pre-treatment of the catalyst with an inert gas comprising water or alcohol vapour hence leads to a catalyst with a higher selectivity towards dimers, e.g. contacting isobutene with the pre-treated catalyst composition will result in a high selectivity towards isooctene (2,4,4-Trimethyl pentene). It is also advantageous that the feed stream does not require the presence of additive(s)/hydrating agent(s) which leads to the production of unwanted product(s).

The contact temperature of the zeolite with the inert gas comprising water vapour or alcohol vapour is preferably 30-90° C., more preferably 40-70° C.

The inert gas preferably comprises water vapour or alcohol vapour in an amount of at least 50 wt % of the saturation level of water vapour or the alcohol vapour in the inert gas, respectively. Preferably, the amount of the water vapour or alcohol vapour is at least 70 wt %, more preferably at least 80 wt %, more preferably at least 90 wt %, more preferably at least 95 wt %, more preferably at least 99 wt % of the saturation level of the water vapour or alcohol vapour in the inert gas, respectively. The inert gas is most preferably saturated with water vapour or alcohol vapour.

Preferably, the inert gas preferably comprises 2-3.2 wt %, more preferably 2.5-3.0 wt % of water vapour.

The alcohol is preferably selected from the group consisting of 1-propanol, isopropanol, isobutanol and tertiary butanol or a mixture thereof.

The inert gas is preferably selected from the group consisting of nitrogen, helium and argon or a mixture thereof.

In particularly preferred embodiments, the inert gas comprises 2-3.2 wt % of water vapour and the inert gas is nitrogen.

Optional Contacting with Dry Gas

The pre-treatment method may further comprise the step of contacting the zeolite with an inert gas which does not comprise water vapour or alcohol vapour (hereinafter sometimes referred as “dry inert gas” or “dry gas”) before and/or after the contacting step with the inert gas comprising water or alcohol vapour. The contacting step with the dry inert gas may be performed at the same temperature as or different temperature from the temperature of the contacting step with the inert gas comprising water vapour or alcohol. For example, the contacting step with the dry gas may be performed at 30 to 350° C. The duration of the contacting step with the dry gas may e.g. be 0.1 to 3 hours.

Contacting with Dry Gas Before Contacting with the Moist Gas

The contacting step with the dry inert gas before the contacting step with the inert gas comprising water or alcohol vapour is preferably performed at a temperature between 100 to 300° C. Contacting the catalyst composition with the dry inert gas at a temperature between 100 to 300° C. before contacting with the inert gas comprising water or alcohol vapour helps quick removal of pre-adsorbed gases from the catalyst, resulting in cleaning the catalyst surface.

The contacting step with the dry inert gas before the contacting step with the inert gas comprising water or alcohol vapour may e.g. be 0.3 to 1.8 hours. It was found that this range is suitable for removing pre-adsorbed gases from the catalyst.

Contacting with the Moist Gas

The contacting step with the inert gas comprising water or alcohol vapour is performed at a temperature between 30° C. and the boiling temperature of the water or the alcohol, preferably 30-90° C., more preferably 40-70° C. In other words, if the inert gas is water vapour, then the contacting step with the inert gas is performed at a temperature between 30° C. to the boiling temperature of the water, preferably 30-90° C., more preferably 40-70° C. In other words, if the inert gas is alcohol vapour, then the contacting step with the inert gas is performed at a temperature between 30° C. to the boiling temperature of the alcohol, preferably 30-90° C., more preferably 40-70° C.

The contacting step with the inert gas comprising water or alcohol vapour is preferably performed for 0.2 to 2.0 hours. It was found that this range results in a combination of good isobutene conversion rate and good selectivity. Particularly preferred is contacting for 0.4 to 1.2 hours or 0.6 to 1.0 hours, which results in a combination of good conversion rate and very high selectivity towards dimer.

Contacting with Dry Gas after Contacting with the Moist Gas

The contacting step with the dry inert gas after the contacting step with the inert gas comprising water or alcohol vapour is preferably performed at a temperature between 50° C. and the boiling temperature of water or the alcohol. Particularly preferred is the contacting at the boiling temperature of water or the alcohol or at a temperature at most 10° C. lower than the boiling temperature of water or the alcohol, which results in a combination of good conversion and very high selectivity towards dimer.

The contacting step with the dry inert gas after the contacting step with the inert gas comprising water or alcohol vapour may e.g. be 0.1 to 1.5 hours. Particularly preferred is contacting for 0.5 to 1.0 hours, which results in a combination of good conversion and very high selectivity towards dimer.

Particularly preferred embodiments include a pre-treatment method comprising contacting with the dry gas at 300° C. for 1 h, subsequently contacting with the moist gas at 50° C. for 1 h, and subsequently contacting with the dry gas at the boiling temperature of water or the alcohol for 0.5 h.

### Zeolite

The zeolite used in the present invention is crystalline materials. Crystalline materials are preferred because of their regular pore size and channelling framework structures.

As used herein, the term “zeolite” or “aluminosilicate zeolite” relates to an aluminosilicate molecular sieve. These inorganic porous materials are well known to the skilled person. An overview of their characteristics is for example provided by the chapter on Molecular Sieves in Kirk-Othmer Encyclopedia of Chemical Technology, Volume 16, p 811-853; in Atlas of Zeolite Framework Types, 5th edition, (Elsevier, 2001).

Aluminosilicate zeolites are generally characterized by the Si/Al ratio of the framework. This ratio may vary widely in the catalyst composition used in the method according to the invention. Preferably, the silicon to aluminium (Si:Al) molar ratio of the zeolite is from about 5 to 1000, more preferably from about 8 to 500.

More preferably, the Si:Al molar ratio of the zeolite is 10-100, preferably 30-60 and most preferably 40-50. This molar ratio of the zeolite is very useful as the zeolite with Si/Al ratio in this range contains moderate acidity and assists in promoting acid catalyzed isobutene dimerization process selectively. This is achieved mostly by preventing unwanted oligomerization reaction through suppressing the severity of the reaction.

Any aluminosilicate that shows activity in the dimerization of olefins may be applied. Examples of suitable materials include the mordenite framework inverted (MFI) and other zeolite structures known to the skilled person, for example MEL, MWW, BEA, MOR, LTL and MTT type. Preferred materials are those known as ZSM-5, ZSM-11, ZSM-8, ZSM-12, ZSM-22, ZSM-23, ZSM-35, ZSM-38, and beta. Most preferably the zeolite is a MFI type zeolite, for example a ZSM-5 zeolite.

The term “medium pore zeolite” is commonly used in the field of zeolite catalyst compositions. A medium pore size zeolite is a zeolite having a pore size of 5-6 Å. Preferably, the zeolite has a pore size of 5-6 Å, more preferably a pore size of 5.2-5.8 Å. Medium pore size zeolites are 10-ring zeolites. i.e. the pore is formed by a ring consisting of 10 SiO₄ tetrahedra. Zeolites of the 8-ring structure type are called small pore size zeolites; and those of the 12-ring structure type, like for example beta zeolite, are referred to as large pore sized. In the above cited Atlas of Zeolite Framework Types, various zeolites are listed based on ring structure. Preferably, the zeolite is a medium pore size aluminosilicate zeolite.

The zeolite used in the present invention may be dealuminated. Means and methods to obtain dealuminated zeolite are well known in the art and include, but are not limited to the acid leaching technique; see e.g. Post-synthesis Modification I; Molecular Sieves, Volume 3; Eds. H. G. Karge, J. Weitkamp; Year (2002); Pages 204-255. Preferably, the zeolite is a dealuminated zeolite having a SiO₂ to Al₂O₃ molar ratio of 10 to 200, for improving the performance/stability of the catalyst composition. Means and methods for quantifying the SiO₂ to Al₂O₃ molar ratio of a dealuminated zeolite are well known in the art and include, but are not limited to AAS (Atomic Absorption Spectrometer) or ICP (Inductively Coupled Plasma Spectrometry) analysis.

The zeolite used in the present invention is in the hydrogen form: i.e. having at least a portion of the original cations associated therewith replaced by hydrogen. Methods to convert an aluminosilicate zeolite to the hydrogen form are well known in the art. One method involves base-exchange using ammonium salts followed by calcination.

The zeolite used in the present invention may comprise up to 5 wt-% of one or more elements selected from Groups 6 and 9 of the Periodic Table, i.e. chromium, molybdenum, tungsten, seaborgium, cobalt, rhodium, iridium and meitnerium. This results in an even higher selectivity towards isooctene. Preferably, said one or more elements are selected from the group consisting of molybdenum, tungsten, cobalt and rhodium.

The amount of these elements in the zeolite may e.g. be less than or equal to 5 wt-%, less than or equal to 4 wt-%, less than or equal to 3-wt %, less than or equal to 2-wt %, less than or equal to 1.5 wt %, less than or equal to 1.25 wt %, less than or equal to 1.1 wt %, or less than or equal to 1.0 wt %. The amount of these elements in the zeolite may e.g. be greater than or equal to 0.05 wt %, greater than or equal to 0.1 wt %, greater than or equal to 0.25 wt %, or greater than or equal to 0.5 wt %. For example, the amount of these elements in the zeolite may be 0.05 wt % to 5 wt %, or 0.25 wt % to 4 wt %.

These elements may be present in the zeolite structure as framework or non-framework element; as counter ion in the zeolite; on its surface, e.g. in the form of metal oxides; or be present in a combination of these forms.

Preferably, the catalyst used in present invention has the framework structure of ZSM-5 with Brønsted acid sites provided by tetrahedrally coordinated aluminium in the framework structure and Lewis and/or Brønsted acid sites provided by elements from Groups 6 and 9 of the Periodic table in the framework structure.

The zeolite catalyst used in the present invention can be prepared by suitable methods of preparing and modifying zeolites as well known to the skilled person; including for example impregnation, calcination, steam and/or other thermal treatment steps. Such methods are disclosed for instance in documents U.S. Pat. No. 7,186,872B2; U.S. Pat. No. 4,822,939 and U.S. Pat. No. 4,180,689 hereby incorporated by reference. The zeolite catalyst used in the present process can also be made by ion exchange technique, sonification technique, precipitation technique, which are all well known to the skilled person.

The catalyst composition comprises a zeolite catalyst as described above. The catalyst composition may consist of the zeolite catalyst as described above, or the catalyst composition may comprise further components such as diluents or binders or other support materials. Preferably these further components do not negatively affect the catalytic performance of the catalyst composition of the invention. Such components are known to the skilled person.

For example, the catalyst composition of the invention may further comprise a non-acidic inert diluent. Preferably the non-acidic inert diluent is silica.

Examples of suitable binder materials include metal oxides, mixed metal oxides, clays, metal carbides and metal oxide hydroxides. The metal oxide or the mixed metal oxides may be chosen from the group of metal oxides comprising for example, oxides of magnesium, aluminium, titanium, zirconium and silicon. The clay may be, but is not limited to, kaolin, montmorillonite or betonite. Metal carbides suitable for use in the composition of the invention are, for example, molybdenum carbide and silicon carbide. The metal oxide hydroxide may be feroxyhyte or Goethite, or more preferably boehmite.

The binder may be present in the composition according to the invention in for example at least 5 wt %, for example at least 10 wt %, for example at least 20 wt %, for example at least 30 wt %, for example at least 40 wt %, for example at least 50% and/or for example at most 5 wt %, for example at most 10 wt %, for example at most 20 wt %, for example at most 30 wt %, for example at most 40 wt %, for example at most 50 wt % with respect to the total catalyst composition.

If the zeolite catalyst composition is to contain a binder, such catalyst composition can be obtained, for example, by mixing the zeolite and a binder in a liquid, and forming the mixture into shapes, like pellets or tablets, applying methods known to the skilled person.

A further aspect of the present invention provides the pre-treated catalyst obtainable by the method according to the present invention.

A further aspect of the present invention provides a process for the dimerization of olefins or a mixture of olefins and paraffins, said olefins and paraffins having between 2 to 10 carbon atoms, preferably 2 to 8 carbon atoms, comprising:

(a) pre-treating a catalyst composition according to the method according to the present invention; and
(b) contacting the treated catalyst composition with a feedstream comprising said olefins or a mixture of olefins and paraffins.

A high selectivity to dimer was achieved. Catalyst was found to be stable.

Preferably, the molar ratio of the olefins to paraffins in the feedstream is 1:0.1-10, more preferably 1:0.2-5, and most preferably 1:0.5-2.

Preferably, the feedstream comprises isobutene and isobutane. The present invention is especially advantageous in this case since isobutene dimerization occurs at low temperature and the reaction is very sensitive to the acidity/acid strength of the zeolite. It is hence important to control acidity of the catalyst and the contact of the feed to the catalyst for the dimerization of isobutene.

The feed stream may further contain one or more diluents, the concentration of which may vary over wide ranges; preferably the feed stream comprises 10-90 vol % of a feed diluent. Examples of suitable diluents include helium, nitrogen, carbon dioxide, and water.

The step of contacting the feed stream with the treated catalyst composition can be performed in any suitable reactor, as known to a skilled man, for example in a fixed bed, a fluidized bed, or any other circulating or moving bed reactor.

With reactor is meant a device for containing and controlling a chemical reaction, in this case dimerization reaction of olefins such as isobutene.

The step of contacting the feed stream with the treated catalyst composition is performed at olefin dimerization conditions. These conditions are known from the prior art. A higher temperature generally enhances conversion and formation of oligomers. However, higher temperatures may induce side-reactions or promote deactivation of the catalyst. Therefore, the contacting step is preferably performed at a temperature of 40-80° C.

Suitable pressures to conduct the contacting step are from between 1-2.5 MPa.

The flow rate at which the feed stream is fed to the reactor may vary widely, but is preferably such that a liquid hourly space velocity (LHSV) results of about 0.1-100 h⁻¹, more preferably LHSV is about 0.5-50 h⁻¹, or 1-20 h⁻¹ or most preferably 7.5-15 h⁻¹. The LHSV may be preferably at least 0.1 h⁻¹, for example at least 10 h⁻¹, for example at least 20 h⁻¹, for example at least 30 h⁻¹ and/or for example at most 1 h⁻¹, for example at most 10 h⁻¹, for example at most 20 h⁻¹, for example at most 30 h⁻¹, for example at most 40 h⁻¹, for example at most 50 h⁻¹. LHSV is the ratio of the rate at which the feed stream is fed to the reactor (in volume per hour) divided by the weight of catalyst composition in a reactor; and is thus inversely related to contact time. By contact time is meant the period of time during which the feedstream is in contact with the catalyst composition.

The LHSV indicates that there is a certain rate at which the feedstream is fed to the reactor. The total length of time in which the feedstream is fed to the reactor is known as the “Time-on-Stream (TOS).” The TOS may be for example at least 2 hours, for example at least 10 hours, for example at least 50 hours, for example at least 100 hours and/or for example at most 2 hours, for example at most 10 hours, for example at most 50 hours, for example at most 100 hours. For example the TOS for a catalyst composition according to the invention during which time the catalyst composition maintains its activity in terms of a high conversion and high selectivity for isooctene, ranges from for example 10 to 100 hours, for example from 15 to 50 hours.

Although the invention has been described in detail for purposes of illustration, it is understood that such detail is solely for that purpose and variations can be made therein by those skilled in the art without departing from the spirit and scope of the invention as defined in the claims.

It is further noted that the invention relates to all possible combinations of features described herein, preferred in particular are those combinations of features that are present in the claims.

It is further noted that the term ‘comprising’ does not exclude the presence of other elements. However, it is also to be understood that a description on a product comprising certain components also discloses a product consisting of these components. Similarly, it is also to be understood that a description on a process comprising certain steps also discloses a process consisting of these steps.

The invention will now be further illustrated with below described experiments.

### EXAMPLES

### Comparative Experiment 1

Various pre-treated catalysts were used for isobutene dimerization, as shown in Table 1.

Pre-calcined samples as shown in Table 1 were pre-treated at 100° C. for 1 h under 20 ml/min of flowing dry N₂.

A feed of isobutene diluted with isobutane (50:50 weight ratio) was contacted with the pre-treated catalysts at 50° C. and 20 bar for 2.5 hours.

TABLE 1

| | | Selectivity/% | | | |
|---|---|---|---|---|---|
| | | C₈ | | | |
| | Isobutene | 2,4,4- | | | |
| Catalysts | con- | Trimethyl- | | | |
| (Si/Al ratio) | version/% | pentene | Others | C₁₂ | C_{16 & +} |
| H-ZSM-5 (27.5) | 99.8 | 13.4 | 1.2 | 43.0 | 42.4 |
| H-ZSM-5 (45) | 37.4 | 87.9 | 0.0 | 4.3 | 7.8 |
| H-Mordenite (20) | 98.0 | 28.0 | 8.3 | 49.5 | 14.2 |
| H-Beta (20.5) | 93.5 | 16.1 | 0.2 | 62.5 | 21.2 |
| H-Y (6) | 77.3 | 12.9 | 0.5 | 27.6 | 59.0 |
| H-SAPO-5 (0.4) | 10.3 | 79.0 | 0 | 17.5 | 3.5 |
| H-SAPO-11 (0.1) | 0.4 | 100 | 0 | 0 | 0 |

### Comparative Experiment 2

Results of isobutene dimerization over H-ZSM-5 zeolite having different Si/Al ratios are shown in Table 2. The pre-treatment of the catalyst, the feed and the dimerization conditions were the same as in comparative experiment 1.

TABLE 2

| | | Selectivity/% | | | |
|---|---|---|---|---|---|
| | | C₈ | | | |
| | Isobutene | 2,4,4- | | | |
| Si/Al mole | con- | Trimethyl- | | | |
| ratio | version/% | pentene | Others | C₁₂ | C_{16 & +} |
| 27.5 | 99.8 | 13.4 | 1.2 | 43.0 | 42.4 |
| 35 | 61.2 | 81.5 | 0.0 | 11.7 | 6.8 |
| 45 | 37.4 | 87.9 | 0.0 | 4.3 | 7.8 |
| 100 | 14.6 | 93.9 | 0.0 | 6.1 | 0.0 |

### Comparative Experiment 3

H-ZSM-5 zeolite (Si/Al ratio 45) catalysts pre-treated at different conditions were used for isobutene dimerization.

Pre-calcined samples were pre-treated at various temperatures shown in Table 3 for 1 h under 20 ml/min of flowing dry N₂.

The feed and the dimerization conditions were the same as in comparative experiment 1.

TABLE 3

| | | Selectivity/% | | | |
|---|---|---|---|---|---|
| | | C₈ | | | |
| Catalysts | Isobutene | 2,4,4- | | | |
| pre-treatment | con- | Trimethyl- | | | |
| temperature/° C. | version/% | pentene | Others | C₁₂ | C_{16 & +} |
| 50 | 35.9 | 89.3 | 1.0 | 8.9 | 0.8 |
| 100 | 37.4 | 87.9 | 0.0 | 4.3 | 7.8 |
| 120 | 72.1 | 81.3 | 0.4 | 16.5 | 1.8 |
| 150 | 94.6 | 35.7 | 9.4 | 48.2 | 6.7 |
| 200 | 98.4 | 10.7 | 32.7 | 53.9 | 2.7 |
| 300 | 99.5 | 3.4 | 5.7 | 28.3 | 62.6 |

### Example 1

Various pre-treated catalysts shown in Table 4 was used for isobutene dimerization.

Pre-calcined samples as shown in Table 4 was pre-treated at 300° C. for 1 h under dry N₂, subsequently 50° C. for 1 h under moist N₂ comprising around 3 wt % of water vapour, subsequently 100° C. for 0.5 h under dry N₂.

The dimerization conditions were the same as in comparative experiment 1.

TABLE 4

| | | Selectivity/% | | | |
|---|---|---|---|---|---|
| | | C₈ | | | |
| | Isobutene | 2,4,4- | | | |
| Catalysts | con- | Trimethyl- | | | |
| (Si/Al ratio) | version/% | pentene | Others | C₁₂ | C_{16 & +} |
| H-ZSM-5 (27.5) | 72.8 | 39.4 | 0.2 | 36.8 | 23.6 |
| H-ZSM-5 (45) | 47.0 | 88.9 | 0.6 | 8.4 | 2.1 |
| H-Mordenite (20) | 87.4 | 44.3 | 3.9 | 41.3 | 10.5 |
| H-Beta (20.5) | 83.0 | 31.4 | 0.0 | 44.5 | 24.1 |
| H-Y (6) | 68.2 | 34.0 | 0.1 | 19.6 | 46.3 |
| H-SAPO-5 (0.4) | 18.5 | 83.3 | 0 | 16.7 | 0.0 |

Comparative experiment 1 in which moist gas is not used results in low C₈ selectivity. Comparison of Tables 1 and 4 shows that the contacting with an inert gas comprising water vapour results in a higher selectivity of the desired 2,4,4-trimethyl pentene.

### Example 2

H-ZSM-5 zeolite (Si/Al ratio 45) catalysts pre-treated at different conditions were used for isobutene dimerization.

Pre-calcined samples were pre-treated at various conditions including the contacting step with moist N₂ comprising around 3 wt % of water vapour. The duration of the contacting step with dry N₂ before the contacting step with moist N₂ was varied as shown in Table 5.

The feed and the dimerization conditions were the same as in comparative experiment 1.

TABLE 5

| | | Selectivity/% | | | |
|---|---|---|---|---|---|
| | | C₈ | | | |
| Moisture pre-treatment | Isobutene | 2,4,4- | | | |
| conditions/Setp-1/ | con- | Trimethyl- | | | |
| Step-2/Step-3 | version/% | pentene | Others | C₁₂ | C_{16 & +} |
| 300° C. for 0.5 h | 38.8 | 92.8 | 0.0 | 6.2 | 1.0 |
| under dry N₂/ | | | | | |
| 50° C. for 1 h | | | | | |
| under moist N₂/ | | | | | |
| 100° C. for 0.5 h | | | | | |
| under dry N₂ | | | | | |
| 300° C. for 1 h | 47.0 | 88.9 | 0.6 | 8.4 | 2.1 |
| under dry N₂/ | | | | | |
| 50° C. for 1 h | | | | | |
| under moist N₂/ | | | | | |
| 100° C. for 0.5 h | | | | | |
| under dry N₂ | | | | | |
| 300° C. for 1.5 h | 47.4 | 87.4 | 0.6 | 9.7 | 2.3 |
| under dry N₂/ | | | | | |
| 50° C. for 1 h | | | | | |
| under moist N₂/ | | | | | |
| 100° C. for 0.5 h | | | | | |
| under dry N₂ | | | | | |

### Example 3

H-ZSM-5 zeolite (Si/Al ratio 45) catalysts pre-treated at different conditions were used for isobutene dimerization.

Pre-calcined samples were pre-treated at various conditions including the contacting step with moist N₂ comprising around 3 wt % of water vapour. The duration of the contacting step with moist N₂ was varied as shown in Table 6.

The feed and the dimerization conditions were the same as in comparative experiment 1.

TABLE 6

| | | Selectivity/% | | | |
|---|---|---|---|---|---|
| | | C₈ | | | |
| Moisture | Isobutene | 2,4,4- | | | |
| pre-treatment | con- | Trimethyl- | | | |
| period/h | version/% | pentene | Others | C₁₂ | C_{16 & +} |
| 300° C. for 1 h under dry N₂/ | 84.8 | 82.6 | 0.5 | 16.1 | 0.8 |
| 50° C. for 0.5 h under moist N₂/ | | | | | |
| 100° C. for 0.5 h under dry N₂ | | | | | |
| 300° C. for 1 h under dry N₂/ | 47.0 | 88.9 | 0.6 | 8.4 | 2.1 |
| 50° C. for 1 h under moist N₂/ | | | | | |
| 100° C. for 0.5 h under dry N₂ | | | | | |
| 300° C. for 1 h under dry N₂/ | 45.1 | 89.2 | 0.5 | 9.0 | 1.3 |
| 50° C. for 1.5 h under moist N₂/ | | | | | |
| 100° C. for 0.5 h under dry N₂ | | | | | |

### Example 4

H-ZSM-5 zeolite (Si/Al ratio 45) catalysts pre-treated at different conditions were used for isobutene dimerization.

Pre-calcined samples were pre-treated at various conditions including the contacting step with moist N₂ comprising around 3 wt % of water vapour. The temperature of the contacting step with dry N₂ after the contacting step with moist N₂ was varied as shown in Table 7.

The feed and the dimerization conditions were the same as in comparative experiment 1.

TABLE 7

| | | Selectivity/% | | | |
|---|---|---|---|---|---|
| | | C₈ | | | |
| | Isobutene | 2,4,4- | | | |
| Moisture removal | con- | Trimethyl- | | | |
| temperature/° C. | version/% | pentene | Others | C₁₂ | C_{16 & +} |
| 300° C. for 1 h under dry N₂/ | 42.4 | 93.0 | 0.8 | 4.6 | 1.6 |
| 50° C. for 1 h under moist N₂/ | | | | | |
| 75° C. for 0.5 h under dry N₂ | | | | | |
| 300° C. for 1 h under dry N₂/ | 47.0 | 88.9 | 0.6 | 8.4 | 2.1 |
| 50° C. for 1 h under moist N₂/ | | | | | |
| 100° C. for 0.5 h under dry N₂ | | | | | |
| 300° C. for 1 h under dry N₂/ | 78.2 | 82.4 | 0.5 | 15.8 | 1.3 |
| 50° C. for 1.5 h under moist N₂/ | | | | | |
| 125° C. for 0.5 h under dry N₂ | | | | | |

### Example 5

H-ZSM-5 zeolite (Si/Al ratio 45) catalysts pre-treated at different conditions were used for isobutene dimerization.

Pre-calcined samples were pre-treated at various conditions including the contacting step with moist N₂ comprising around 3 wt % of water vapour. The duration of the contacting step with dry N₂ after the contacting step with moist N₂ was varied as shown in Table 8.

The feed and the dimerization conditions were the same as in comparative experiment 1.

TABLE 8

| | | Selectivity/% | | | |
|---|---|---|---|---|---|
| | | C₈ | | | |
| | Isobutene | 2,4,4- | | | |
| Moisture removal | con- | Trimethyl- | | | |
| period/h | version/% | pentene | Others | C₁₂ | C_{16 & +} |
| 300° C. for 1 h under dry N₂/ | 34.3 | 90.0 | 0.8 | 7.6 | 1.6 |
| 50° C. for 1 h under moist N₂/ | | | | | |
| 100° C. for 0.25 h under dry N₂ | | | | | |
| 300° C. for 1 h under dry N₂/ | 47.0 | 88.9 | 0.6 | 8.4 | 2.1 |
| 50° C. for 1 h under moist N₂/ | | | | | |
| 100° C. for 0.5 h under dry N₂ | | | | | |
| 300° C. for 1 h under dry N₂/ | 55.0 | 88.1 | 0.8 | 11.1 | 0.0 |
| 50° C. for 1 h under moist N₂/ | | | | | |
| 100° C. for 0.75 h under dry N₂ | | | | | |
| 300° C. for 1 h under dry N₂/ | 64.2 | 87.6 | 0.5 | 11.5 | 0.4 |
| 50° C. for 1 h under moist N₂/ | | | | | |
| 100° C. for 1 h under dry N₂ | | | | | |

### Example 6

Effect of reaction temperature on the performance of H-ZSM-5 zeolite (Si/Al ratio of 45) for isobutene dimerization is shown in table 9.

The feed and the reaction conditions, except temperature, were the same as in comparative experiment 1.

Catalyst pre-treatment: Pre-calcined sample was treated at 300° C. for 1 h under dry N₂/50° C. for 1 h under moist N₂ comprising around 3 wt % of water vapour/100° C. for 0.5 h under dry N₂.

TABLE 9

| | | Selectivity/% | | | |
|---|---|---|---|---|---|
| | | C₈ | | | |
| | Isobutene | 2,4,4- | | | |
| Reaction | con- | Trimethyl- | | | |
| temperature/° C. | version/% | pentene | Others | C₁₂ | C_{16 & +} |
| 50 | 47.0 | 88.9 | 0.6 | 8.4 | 2.1 |
| 60 | 52.0 | 84.2 | 0.8 | 14.2 | 0.8 |
| 70 | 78.6 | 63.9 | 0.3 | 29.3 | 6.5 |

### Example 7

Effect of feed composition on the performance of H-ZSM-5 zeolite (Si/Al ratio of 45) for isobutene dimerization is shown in table 10.

Reaction conditions were the same as in comparative experiment 1.

Catalyst pre-treatment: Pre-calcined sample was treated at 300° C. for 1 h under dry N₂/50° C. for 1 h under moist N₂ comprising around 3 wt % of water vapour/100° C. for 0.5 h under dry N₂.

TABLE 10

| | | Selectivity/% | | | |
|---|---|---|---|---|---|
| | | C₈ | | | |
| Isobutene/ | Isobutene | 2,4,4- | | | |
| Isobutane/ | con- | Trimethyl- | | | |
| Weight ratio | version/% | pentene | Others | C₁₂ | C_{16 & +} |
| 40:60 | 47.9 | 90.8 | 0.5 | 8.7 | 0.0 |
| 50:50 | 47.0 | 88.9 | 0.6 | 8.4 | 2.1 |
| 60:40 | 71.5 | 73.3 | 3.3 | 19.7 | 3.7 |

Set forth below are some embodiments of the process and catalyst composition.

### Embodiment 1

A method for pre-treating a catalyst composition comprising: contacting a medium pore aluminosilicate zeolite with an inert gas comprising water vapour or alcohol vapour at a temperature between 30° C. and the boiling temperature of the water or the alcohol.

### Embodiment 2

The method according to Embodiment 1, wherein the medium pore aluminosilicate zeolite is contacted with an inert gas at a temperature of 30-90° C.

### Embodiment 3

The method according to any one of Embodiments 1-2, wherein the medium pore aluminosilicate zeolite is contacted with an inert gas at a temperature of 40-70° C.

### Embodiment 4

The method according to any one of Embodiments 1-3, wherein the alcohol is at least one selected from 1-propanol, isopropanol, isobutanol, tertiary butanol, and a mixture thereof.

### Embodiment 5

The method according to any one of Embodiments 1-4, wherein the inert gas is at least one selected from nitrogen, helium, argon, and a mixture thereof.

### Embodiment 6

The method according to any one of Embodiments 1-5, wherein the zeolite is a 10-ring zeolite having pores formed by a ring consisting of 10 SiO₄ tetrahedra.

### Embodiment 7

The method according to any one of Embodiments 1-6, wherein the zeolite has a pore size of 5-6 Å.

### Embodiment 8

The method according to any one of Embodiments 1-7, wherein the zeolite has a pore size of 5.2-5.8 Å.

### Embodiment 9

The method according to any one of Embodiments 1-8, the zeolite is of the ZSM-5 type.

### Embodiment 10

The method according to any one of Embodiments 1-9, wherein the silicon to aluminium (Si:Al) molar ratio of the zeolite is 10-100.

### Embodiment 11

The method according to any one of Embodiments 1-10, wherein the silicon to aluminium (Si:Al) molar ratio of the zeolite is 30-60.

### Embodiment 12

The method according to any one of Embodiments 1-11, wherein the silicon to aluminium (Si:Al) molar ratio of the zeolite is 40-50.

### Embodiment 13

The method according to any one of Embodiments 1-712 wherein the zeolite comprises up to 1 wt-% of at least one element selected from Groups 6 and 9 of the Periodic Table.

### Embodiment 14

The method according to Embodiment 13, wherein the element is at least one selected from molybdenum, tungsten, cobalt, and rhodium.

### Embodiment 15

The method according to any one of Embodiments 1-14, wherein the amount of the vapour in the inert gas is at least 40 wt % of the saturation level of the vapour in the inert gas, wherein the vapour is water vapour or alcohol vapour.

### Embodiment 16

The method according to any one of Embodiments 1-15, wherein the amount of the vapour in the inert gas is at least 50 wt % of the saturation level of the vapour in the inert gas, wherein the vapour is water vapour or alcohol vapour.

### Embodiment 17

The method according to any one of Embodiments 1-16, wherein the amount of the vapour in the inert gas is at least 60 wt % of the saturation level of the vapour in the inert gas, wherein the vapour is water vapour or alcohol vapour.

### Embodiment 18

A pre-treated catalyst composition obtainable by the method according to any one of Embodiments 1-17.

### Embodiment 19

A process for the dimerization of olefins or a mixture of olefins and paraffins, said olefins and paraffins having between 2 to 8 carbon atoms, comprising: contacting the pre-treated catalyst composition of Embodiment 16 with a feedstream comprising said olefins or a mixture of olefins and paraffins.

### Embodiment 20

The process according to Embodiment 19, wherein the molar ratio of the olefins to paraffins in the feedstream is 1:0.1-10.

### Embodiment 21

The process according to Embodiment 19, wherein the molar ratio of the olefins to paraffins in the feedstream is 1:0.2-5.

### Embodiment 22

The process according to Embodiment 19, wherein the molar ratio of the olefins to paraffins in the feedstream is 1:0.5-2.

### Embodiment 23

The process according to any one of Embodiments 19-22, wherein the feedstream comprises isobutene and isobutane.

### Embodiment 24

The process according to any one of Embodiments 19-23, wherein the pre-treated catalyst composition is contacted with a feedstream under conditions comprising a temperature of 40-80° C. and a pressure of 1-2.5 MPa.

What is claimed is:

1. A method for pre-treating a catalyst composition comprising: contacting a medium pore aluminosilicate zeolite with an inert gas comprising water vapour or alcohol vapour at a temperature between 30° C. and the boiling temperature of water or the alcohol to form the pre-treated catalyst.

2. The method according to claim 1, wherein the alcohol is at least one selected from 1-propanol, isopropanol, isobutanol, tertiary butanol, and a mixture thereof.

3. The method according to claim 2, wherein the inert gas is at least one selected from nitrogen, helium, argon, and a mixture thereof.

4. The method according to claim 3, wherein the zeolite is a 10-ring zeolite having pores formed by a ring consisting of 10 SiO₄ tetrahedra.

5. The method according to claim 4, wherein the zeolite has a pore size of 5-6 Å.

6. The method according to claim 5, wherein the zeolite is of the ZSM-5 type.

7. The method according to claim 6, wherein the silicon to aluminium (Si:Al) molar ratio of the zeolite is 10-100.

8. The method according to claim 7, wherein the zeolite comprises up to 1 wt-% of at least one element selected from Groups 6 and 9 of the Periodic Table.

9. The method according to claim 8, wherein the element is at least one selected from molybdenum, tungsten, cobalt, and rhodium.

10. The method according to claim 9, wherein the amount of the water vapour or the alcohol vapour in the inert gas is at least 50 wt % of the saturation level of the water vapour or the alcohol vapour in the inert gas.

11. A pre-treated catalyst composition obtainable by the method according to claim 1.

12. Process for the dimerization of olefins or a mixture of olefins and paraffins, said olefins and paraffins having between 2 to 8 carbon atoms, comprising:
contacting the pre-treated catalyst composition of claim 11 with a feedstream comprising said olefins or a mixture of olefins and paraffins.

13. The process according to claim 12, wherein the molar ratio of the olefins to paraffins in the feedstream is 1:0.1-10.

14. The process according to claim 13, wherein the feedstream comprises isobutene and isobutane.

15. The process according to claim 14, wherein the pre-treated catalyst composition is contacted with a feedstream under conditions comprising a temperature of 40-80° C. and a pressure of 1-2.5 MPa.

16. The method according to claim 1, wherein the temperature is 30° C. to 90° C.

17. The method according to claim 5, wherein the pore size is 5.2-5.8 Å.

18. The method according to claim 7, wherein the molar ratio is 30-60.

19. The process according to claim 13, wherein the molar ratio is 1:0.2-5.
